(19) United States
(12) Patent Application Publication
(10) Pub. No.: US 2008/0260788 A1
(43) Pub. Date: Oct. 23, 2008

(54) Skin Cleaning Composition

(75) Inventors: Christopher Malyszewiez, Northamptonshire (GB); Ian Geoffrey Pears, Northampton (GB); Rae Clyde Levene, Northampton (GB)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: GREENBRIDGE ENVIRONMENTAL CONTROL LIMITED, Bristol (GB)

(21) Appl. No.: 11/569,287

(22) PCT Filed: May 17, 2005
(86) PCT No.: PCT/GB2005/001909

§ 371 Date: Jun. 11, 2008

(30) Foreign Application Priority Data
May 17, 2004 (GB) 0410968.2

(51) Int. Cl. A61K 9/00; A61K 33/18; A61P 17/00; A61P 31/04

(52) U.S. Cl. 424/404; 424/667

(57) ABSTRACT

The present invention describes a skin cleaning and disinfecting composition comprising an active component, wherein the active component comprises at least one alcohol, at least one halogen. The present invention also describes corresponding anti-bacterial, anti-viral and anti-fungal compositions, as well as a method of manufacturing same.

### FIELD OF INVENTION

The present invention relates to a liquid cleansing composition having an anti-viral and/or anti-bacterial action. More particularly, but not exclusively, it relates to a skin cleansing composition having both anti-viral and anti-bacterial activity.

### BACKGROUND

There is an ever expanding number of household products such as handwashes and domestic cleaning sprays professing to provide anti-bacterial properties. Often these products claim to eliminate work surfaces and the like of all known bacteria. Such claims are typically misleading at best. Widely reported research has shown that many of these currently available products are no better at reducing the onset of coughs, colds or other such infections or ailments than thoroughly washing one hands or cleaning the work surfaces. Indeed many such infections or aliments are caused by viruses which currently available anti-bacterial products are unable to combat, despite what they purport to achieve.

There is also an increasing concern about bacterial and viral infections being transmitted to patients and staff in hospitals and the like. One vector of infection is believed to be incompletely disinfected surfaces, which may harbour bacteria and/or viruses that are resistant to existing surface cleaning agents. There is a strong suspicion that the spread of the recent SARS (Severe Acute Respiratory Syndrome) outbreak may have been linked to the ability of the SARS virus to resist conventional cleaning agents/disinfectants. Viruses spread from an infected patient thus remain viable and ready to be picked up by and to infect other patients and medical staff. Other pathogens, such as the MRSA bacterium, are also suspected to be surviving existing surface cleaning/disinfecting agents and routines.

It is known to use cationic surfactants, such as quaternary ammonium salts, as dual-purpose surface cleaning agents and bactericides. However, while such materials are generally found to be sufficient to deal with, say, food-poisoning bacteria in a food preparation environment, they are not regarded as sufficiently active to handle more dangerous and more resistant pathogens in a medical context.

Alcohols, such as iso-propanol, and halogens, such as iodine, have in the past been used individually as relatively crude disinfecting agents around wounds and skin lesions, but they have not proven suitable for wide area cleaning of hard surfaces and the like. For example, iodine can stain many surfaces, and its use at high concentrations is limited by safety considerations.

It is an object of the present invention to provide a liquid cleansing and disinfecting preparation, suitable for use on skin, with a high anti-viral and anti-bacterial effectiveness.

### SUMMARY OF INVENTION

According to a first aspect of the present invention, there is provided a skin cleaning and disinfecting composition comprising an active component, wherein the active component comprises at least one alcohol, at least one long-chain alkyl polyamine, and at least one halogen.

According to a second aspect of the present invention, there is provided a skin cleaning and anti-bacterial composition comprising an active component, wherein the active component comprises at least one alcohol, at least one long-chain alkyl polyamine, and at least one halogen.

According to a third aspect of the present invention, there is provided a skin cleaning and anti-viral composition comprising an active component, wherein the active component comprises at least one alcohol, at least one long-chain alkyl polyamine, and at least one halogen.

According to a fourth aspect of the present invention, there is provided a skin cleaning and anti-fungal composition comprising an active component, wherein the active component comprises at least one alcohol, at least one long-chain alkyl polyamine, and at least one halogen.

According to a fifth aspect of the present invention, there is provided a means of destroying organisms and/or inhibiting the ability of bacteria and/or viruses to replicate when said bacteria and/or viruses are present on skin, the means comprising the application of a composition to said skin wherein the composition is configured to rupture the phospholipid membrane of the bacteria or virus, the composition being further configured to substantially permanently bind to bacterial DNA and viral DNA or RNA. In this context “substantially permanently” is understood to mean that the bacterial DNA or viral DNA or RNA has a component or components of the composition bound thereto such that said DNA or RNA is unable to replicate for at least several hours, but preferably indefinitely. These means advantageously allow for rapid decontamination of skin. Preferably the composition comprises an active component, wherein the active component comprises at least one alcohol, at least one long-chain alkyl polyamine, and at least one halogen. Most preferably the composition is substantially as described in the other aspects of the present invention.

According to a sixth aspect of the present invention, there is provided a means of inhibiting the ability of bacteria and/or viruses to replicate when said bacteria and/or viruses are present on skin, the means comprising the application of a composition to said skin wherein the composition is configured to substantially permanently encapsulate the bacterial or viral structures and prevent the replication of their genetic material. In this context “substantially permanently” is understood to mean that the bacteria or virus has a component or components of the composition bound thereto such that said DNA or RNA is encapsulated to the degree that it is unable to replicate for at least several hours, but preferably indefinitely. These means advantageously allow for rapid decontamination of skin. Preferably the composition comprises an active component, wherein the active component comprises at least one alcohol, at least one long-chain alkyl polyamine, and at least one halogen. Most preferably the composition is substantially as described in the other aspects of the present invention.

It is to be understood within the context of the present invention that the term “active component” does not preclude the composition comprising other constituents that may be considered to be “active” in terms of their chemistry and/or their mode(s) of operation. Rather, the term “active component” is used herein merely as a convenient descriptor for a particular portion of the composition.

In the various aspects of the present invention, the long-chain alkyl polyamine compound of the active component preferably comprises a long-chain alkyl triamine compound and/or a long-chain alkyl tetramine compound. The component may comprise a mixture of long-chain alkyl polyamine compounds having a range of different alkyl chain lengths.

Advantageously, the long-chain alkyl polyamine compound of the active component may comprise a compound of the general formula H₂N(CH₂)₃—NR—(CH₂)₃NH₂, where R is a linear or branched alkyl chain comprising at least eight carbon atoms. R may be a linear or branched alkyl chain comprising between ten and fourteen carbon atoms. Preferably R is a linear alkyl chain comprising twelve carbon atoms.

The active component of the composition preferably comprises between 10% and 30% by volume of the long-chain alkyl polyamine compound or compounds. Advantageously, the component comprises between 15% and 25% of the long-chain alkyl polyamine compound or compounds. Optionally, the component may comprise 20%±2% of the long-chain alkyl polyamine compound or compounds.

Preferably, the at least one aliphatic alcohol of the active component of the composition comprises between one and four carbon atoms. The component preferably comprises two aliphatic alcohols. It is particularly preferred that the composition comprises ethanol and n-propanol.

The active component of the composition may comprise between 10% and 30% by volume of the aliphatic alcohols. Advantageously, the component comprises between 15% and 25% by volume of the aliphatic alcohols. The component may comprise between 10% and 20% by volume ethanol and between 5% and 10% by volume n-propanol. Optionally, the component may comprise between 14% and 16% by volume of ethanol and between 5% and 7% by volume of n-propanol.

Preferably the active component of the composition comprises a mixture of halogens and/or halogen source(s). Alternatively, the component may comprise only a single halogen and/or single halogen source. The preferred halogen of the present invention is iodine, the preferred source of iodine being molecular iodine provided in a solid form.

The component preferably comprises up to 0.5% by weight iodine. Advantageously, the component may comprise between 0.1% and 0.5% by weight iodine. Optionally, the component may comprise 0.33%±0.05% by weight iodine.

The active component of the composition may comprise a complexing agent adapted to form a complex with the halogen.

The active component of the composition may comprise at least one buffering agent, such as nitrilotriacetic acid or its salts.

The active component of the composition may comprise at least one surfactant. Preferably at least one amphoteric surfactant is present in the component. Ideally a mixture of amphoteric surfactants are present in the component.

The active component of the composition may comprise at least one wetting agent, such as a polyglycol ether, optionally a polyethylene glycol ether or a polypropylene glycol ether.

The active component of the composition and the compositions of the present invention are preferably non-dangerous. In this context “non-dangerous” is understood to mean non-dangerous as defined by European Dangerous Preparations Directive (99/45/EC) and the Dangerous Substances Directive (67/548/EEC).

The compositions of the present invention preferably and, in addition to the active component, further comprise at least one surfactant. Preferably said at least one surfactant is amphoteric. Ideally a mixture of amphoteric surfactants are present in the composition.

In a preferred embodiment of the present invention, the composition comprises between 0.01% and 20% by volume of the active component and between 15% and 80% by volume of at least one surfactant, the composition may comprise a % volume of component(s), such as water, to make the composition up to 100%.

Preferably, the composition comprises between 0.1% and 10% by volume of the active component and between 20% and 60% by volume of at least one surfactant. Most preferably however, the composition comprises substantially 1% by volume of the active component and substantially 44% by volume of at least one surfactant, the composition comprising a % volume of component(s), such as water, to make the composition up to 100%.

The composition of the present invention may be provided in a form which is suitable for a number of different forms of delivery to skin. The composition could be provided in a concentrated form for subsequent dilution by a user shortly before being used to clean skin. Once diluted however, the resultant solution may be capable of being stored for up to 12 months and yet still being effective against bacteria and/or viruses and/or fungus and/or a provide a conventional detergent/cleansing effect which removes macroscopic soiling.

The composition of the present invention may be provided in a form ready for immediate delivery to skin. The delivery device for such immediate delivery of the composition may be a controlled spray, such as a trigger spray or the like. Preferably the composition for immediate delivery is capable of being stored in its delivery device for up to 24 months and yet still being effective against bacteria and/or viruses and/or fungus and/or a provide a conventional detergent/cleansing effect which removes macroscopic soiling.

Another delivery device for immediate delivery of the composition of the present invention may be an impregnated cloth wipe. Such wipes could be provided in a container or drum containing numerous wipes, or provided in a single sachet form. Preferably such wipes are capable of being stored in their container for up to 24 months and yet still being effective against bacteria and/or viruses and/or fungus and/or a provide a conventional detergent/cleansing effect which removes macroscopic soiling.

A yet further delivery device for immediate delivery of the composition of the present invention may be a solid bar or tablet. The solid bar or tablet, such as a soap bar, can be formed in the usual manner accustomed to those of skill in the art of solid bar or tablet manufacture.

According to a seventh aspect of the present invention there is provided a method for manufacturing a composition configured for cleaning and disinfecting skin wherein the method comprises: the addition to a pH buffered solution of at least one long-chain alkyl polyamine, to which is added at least one surfactant to make an interim solution; separate to said interim solution a premix solution containing at least one alcohol and at least one halogen is made; the interim solution and the premix solution are then combined, to produce an active component; and wherein the active component is subsequently mixed with at least one surfactant.

According to a eighth aspect of the present invention there is provided a composition configured for use on skin comprising an active component, wherein the active component comprises at least one alcohol, at least one long-chain alkyl polyamine.

According to a ninth aspect of the present invention there is provided a composition configured for use on skin comprising an active component, wherein the active component comprises at least one long-chain alkyl polyamine and at least one halogen.

### SPECIFIC EMBODIMENTS

In order to allow the present invention to be more readily understood an embodiment of the invention will now be described more particularly by way of example only, and with reference to the accompanying drawings in which:

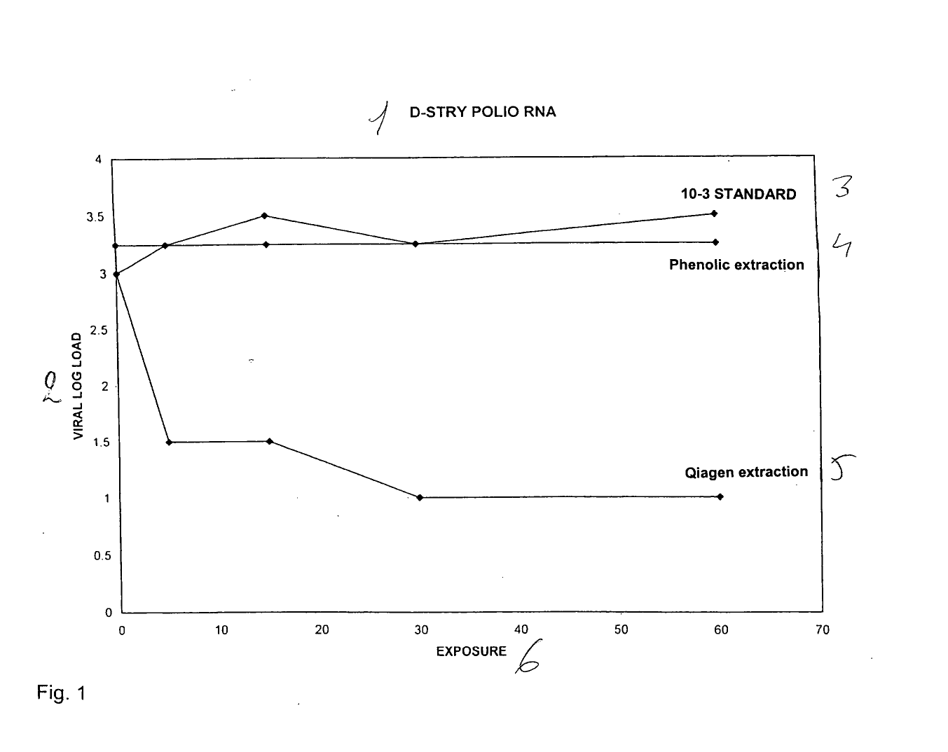

FIG. 1 shows a graph of the composition of the present invention's activity against Polio RNA;

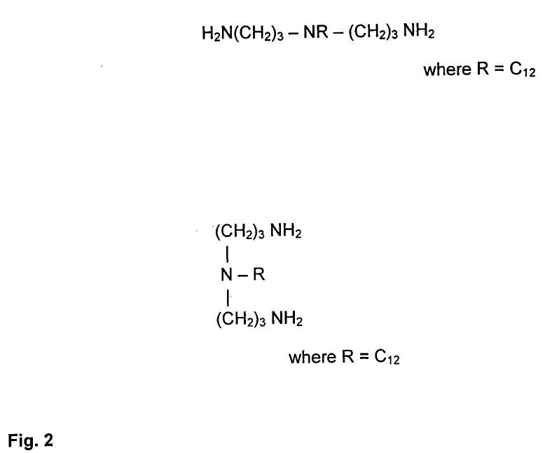

FIG. 2 shows the chemical structure of the long-chain alkyl polyamine of the present invention.

An aqueous surface cleaning composition was prepared by initially preparing the active component, comprising:

| | | |
|---|---|---|
| NTA 89% powder | 0.85 | kg |
| Ethanol | 15.0 | litres |
| n-Propanol | 6.0 | litres |
| Topanol O FG | 0.55 | litres |
| Sandoteric SC | 2.42 | litres |
| Sandozin NRW conc | 6.95 | litres |
| Sandoteric ABD | 4.45 | litres |
| Triameen Y12D-30 | 19.99 | litres |
| Deionised water | 43.4524 | litres |
| Iodine (solid) | 0.3376 | kg |

The active component of the composition appeared as a pale yellow clear liquid with a pH of approximately 8 and a slight alcoholic odour.

NTA is nitrilotriacetic acid trisodium salt, a buffering agent. Topanol O FG is food-grade butylated hydroxytoluene, an antioxidant, sold by Chance & Hunt Ltd. (Topanol is a registered trade mark of ICl plc). Sandozin NRW conc is a polyethoxylate ether sold by Clariant as a wetting agent. It also forms a relatively stable complex with iodine. Sandoteric SC is a sulphobetaine amphoteric surfactant, which acts as a detergent, and Sandoteric ABD is a complex mixture of amphoteric surfactants acting as a detergent and having a degree of bactericidal activity. Both are sold by Clariant. (Sandozin and Sandoteric are registered trade marks of Novartis SA).

Triameen Y12D-30 is a long-chain alkyl triamine of the general fomlula H₂N(CH₂)₃—NR—(CH₂)₃NH₂, where R′ is a “tallow alkyl”—a naturally-derived mixture of alkyl chains of different lengths, the most common of which is a dodecyl chain. It is sold by Akzo Nobel.

The active component was then mixed with the following components to produce the composition of the present invention, namely:

| | |
|---|---|
| Sodium Laureth Sulphate | 40% |
| Cocamidopropyl Betaine | 2% |
| Cocodiethanolamide | 2% |
| First Component | 1% |
| Monopropyl Glycol | 0.5% |
| Fragrance | 0.5% |
| Methyl Paraben | 0.3% |
| Sodium Chloride/ | (As required) |
| Citric Acid (C.I. 42090) | |
| Balance as Water | |

Sodium laureth sulphate, cocamidopropyl betaine, cocodiethanolamide are all surfactants, monopropyl glycol is a solvent/wetting agent, and methyl paraben is a presevative and anti-fungal agent. All of these constituents are widely available commercially.

FIG. 2 illustrates the hypothesised structure of the long-chain alkyl triamine. It is possible that the activity of this molecule is located on the NH₂ groups, the iodine being monotonically bonded to the nitrogen yet accessible toward DNA or RNA. The nucleotides of the DNA or RNA may then be liganded by addition to phosphatide groupings by the presence of the iodated amine group. Thus, the iodine radical may be free to roam on the molecule and as there is partial addition thereof, there is competition for valencey fulfilment.

Although the mechanism of attack by the active component on bacterium is not fully understood, it is expected that in a suitably buffered solution the long-chain alkyl triamine forms a cationic species. Together with the surfactant(s), preferably amphoteric in nature, the triamine attacks the phospholipid membranes which form the outer wall of a bacterium. In most cases, these membranes are ruptured or lysed, leading to release of the bacterium's DNA: It is possible that the complexed halogen and the alcohol(s) act in conjunction on the DNA, effectively complexing with the DNA or RNA. The triamine and the surfactant(s) are believed to attack bacterial DNA and bind to critical parts of the helix preventing it from replicating. The alcohol(s) may also contribute to the attack on the membranes.

Even where the membranes are not sufficiently damaged to release their contents for destruction, the composition is found to inactivate the bacterium for prolonged periods (at least 14 days in current testing, much longer than for current cleaners/disinfectants).

Turning to the mechanism of attack on viruses, similarly this mechanism is also not fully understood. However it is again expected that the cationic triamine formed in a suitably buffered solution attacks the outer wall of the capsid of the virus in conjunction with the surfactant(s), which are preferably amphoteric in nature. It is possible that these structures are ruptured or lysed as a consequence of the attack, leading to release of the viral DNA or RNA. The complexed halogen and the alcohol(s) are believed to act in conjunction on viral DNA or by bonding or associating themselves with parts of the viral RNA. Additionally the triamine and the surfactant(s) are also believed to attack the viral DNA or RNA by binding to critical parts of the helix. The result of the attack(s) on the viral DNA or RNA is the inhibition of the DNA or RNA's ability to replicate. The alcohol(s) may also contribute to the attack on the membranes, particularly the outer viral phospholipid envelope, present in some but not all DNA/RNA viruses.

Alternatively, rather than the viral capsid being ruptured or lysed by the attack of the buffered cationic triamine and the surfactant(s), it is possible that the attack results in the binding to surface structures, blocking and inactivating viral receptors. The result of this attack being the inhibition of infectivity, thus preventing the virus spreading to other cells. It is possible that halogen and the alcohol(s) take some part in the attack of the viral capsid membrane.

Regardless of the mechanism, the combined action of the components of the composition is the break up and destruction of a majority of the organism and/or the inhibition of any viruses or bacteria for prolonged periods. The composition also has a conventional detergent/cleansing effect, removing macroscopic soiling from the skin to which it is applied, as well as washing off inhibited bacteria or viruses as well as the associated debris of the destroyed organism. The composition has been found to have minimal deleterious effect on skin, and does not stain as would conventional formulations containing similar levels of halogen, particularly iodine.

As already mentioned, the mechanism of virucidal action by a composition of the present invention is not clearly understood. From the constituents present in the composition, it is suspected that the alcohol denatures proteins, and the quaternary ammonium compound(s) bind to anionic phosphate groups and fatty acid chains in phospholipids. Both mechanisms damage the microbial membranes. The halogen may modify structural proteins and may inhibit enzymes through halogenation of amino acids in proteins.

Through experimentation, various naked virus or purified animal deoxy-ribose nucleic acid (DNA) samples can be treated with the composition. An interaction takes place, so that when placed in an electric field under gel electrophoresis, a DNA smear is produced instead of the expected DNA ladder of normal integrity, indicating alteration of the ionisation characteristics of DNA. If the same composition/virus or composition/animal DNA mixture is extracted with a mixture of phenol/chloroform, the composition itself is broken down, and the full DNA electrophoretic pattern will be restored with normal integrity. This data indicates that the DNA (viral or animal DNA) is not degraded during the treatment with the composition, but that the composition interaction with viral or animal DNA alters the normal DNA structural and ionic integrity.

The interaction between the composition was further investigated for its affect on viral particles and viral ribose nucleic acid (RNA) and FIG. 1 illustrates the observed activity. Through experimentation where the composition (1 part in 10 parts water) as 9 part diluted composition and 1 part poliovirus vaccine (final 1000 copies/ml) for periods of 5, 15, 30 and 60 minutes, followed by extraction, using QIAGEN silicon columns (QIAGEN incorporates a protease step for protein degradation), prior to complimentary deoxy-ribose nucleic acid (cDNA) synthesis and DNA amplification, (with detection of nucleic acid product in a in-house real-time Lightcycler quantitative RNA assay), reductions in the RNA viral load can be seen from 1000 copies to 50, 50, 10, and 10 copies respectively-(reductions of 95%, 95%, 99%, 99%), compared to 1000, 1000, 1000, 1000 copies/ml respectively in water control samples. The experiment was repeated with composition/virus incubations of 5, 15, 30 and 60 minutes, as previously stated, but RNA extraction performed with a phenol/chloroform procedure (once with phenol, once with 1:1 phenol and chloroform, and once with chloroform rather than by the QIAGEN extraction method), RNA detection will be detected at 1000, 1000, 1000, and 1000 copies respectively (no reduction in RNA load).

The experiments demonstrate that the composition does not degrade RNA over the periods of 1-60 minutes, but that an interaction occurs between the composition and poliovirus/poliovirus RNA. This interaction inhibits protease action (active in the QIAGEN process) to cleave composition peptides that would normally release RNA, or alters naked RNA ionically, so that RNA cannot then be further captured and amplified in the test. The result is a low recovery of RNA, and will appear as a low copy number in the assay. The effect of the composition-virus interaction is removed during the chemical extraction with phenol. It can be concluded that the composition is not destructive to viral capsids, but inhibits enzymatic cleavage of the capsid, and requires further stringent chemical extraction to achieve release of the nucleic acid. The QIAGEN effect illustrated here is a consistent effect and has been replicated for other RNA and DNA viruses including Adenovirus, BK virus and Norovirus, and appears to be a plausible mechanism of virucidal activity, whereby the altered viral structure is resistance to physiological and enzymatic attack.

It can therefore finally concluded that both viral DNA and viral RNA is not degraded by treatment with the composition. However, the composition interacts with the viral capsid and prevents enzymatic cleavage. Although the process of virus uncoating involves different mechanisms for different virus groups, they all require the viral capsid to have structural and ionic integrity and be susceptible to cellular physiological processes that allow it to pass into and through the host cell, and interact with cellular structures and enzyme systems. The changes induced by the composition on viral structures are likely to prevent this process and account for its virucidal activity.

It is hypothesised that compositions with higher levels of halogen may be useful in some applications, although alterations to the other components, such as raised amounts of alcohol(s), may then be needed for stability.

The composition also has a degree of activity against fungi, moulds and yeasts, although it is believed that a modified formulation, for example with an alternative alcohol blend, might be required for full effectiveness against the tougher walls of fungal spore cells and the like.

Testing has shown that the composition passes the standard “555-challenge” test (see British Standard BS EN 1276:1997 and the French Afnor test). As an effective anti-viral and anti-bacterial cleansing agent, it may be categorised as a (2) category disinfectant in the system employed by the UK National Health Service, suitable for cleaning in “medium high risk” areas.

1. A skin cleaning and disinfecting composition comprising an active component, wherein the active component comprises at least one alcohol, at least one long-chain alkyl polyamine, and at least one halogen.

2. A skin cleaning and anti-bacterial composition comprising an active component, wherein the active component comprises at least one alcohol, at least one long-chain alkyl polyamine, and at least one halogen.

3. A skin cleaning and anti-viral composition comprising an active component, wherein the active component comprises at least one alcohol, at least one long-chain alkyl polyamine, and at least one halogen.

4. A skin cleaning and anti-fungal composition comprising an active component, wherein the active component comprises at least one alcohol, at least one long-chain alkyl polyamine, and at least one halogen.

5. The composition according to claim 1, wherein the long-chain alkyl polyamine compound of the active component comprises a long-chain alkyl triamine compound and/or a long chain alkyl tetramine compound.

6. The composition according to claim 1, wherein the long-chain alkyl polyamine compound of the active component comprises a mixture of long-chain alkyl polyamine compounds having a range of different alkyl chain lengths.

7. The composition according to claim 1, wherein the long-chain alkyl polyamine compound of the active component comprises a compound of the general formula H₂N(CH₂)₃—NR—(CH₂)₃NH₂, where R is a linear or branched alkyl chain comprising at least eight carbon atoms.

8. The composition according to claim 7, wherein R is a linear of branched alkyl chain comprising between ten and fourteen carbon atoms.

9. The composition according to claim 7, wherein R is a linear alkyl chain comprising twelve carbon atoms.

10. The composition according to claim 1, wherein the active component of the composition comprises between 10% and 30% by volume of the long-chain alkyl polyamine compound or compounds.

11. The composition according to claim 1, wherein the active component of the composition comprises between 15% and 25% of the long-chain alkyl polyamine compound or compounds.

12. The composition according to claim 1, wherein at least one aliphatic alcohol of the active component of the composition comprises between one and four carbon atoms.

13. The composition according to claim 1, wherein at least one aliphatic alcohol of the active component of the composition comprises at least two aliphatic alcohols.

14. The composition according to claim 13, wherein the active component of the composition comprises between 10% and 30% by volume of the aliphatic alcohols.

15. The composition according to claim 13, wherein the active component of the composition comprises between 15% and 25% by volume of the aliphatic alcohols.

16. The composition according to claim 1, wherein the active component of the composition comprises a single halogen and/or single halogen source.

17. The composition according to claim 1, wherein the active component of the composition comprises iodine.

18. The composition according to claim 1, wherein the active component of the composition up to 0.5% by weight iodine.

19. The composition according to claim 1, wherein the active component of the composition comprises between 0.1% and 0.5% by weight iodine.

20. The composition according to claim 1, wherein the active component of the composition comprises a mixture of halogens and/or halogen source(s).

21. The composition according to claim 1, wherein the active component of the composition comprises a complexing agent adapted to form a complex with the halogen.

22. The composition according to claim 1, wherein the active component of the composition comprises at least one buffering agent.

23. The composition according to claim 1, wherein the active component of the composition comprises at least one surfactant.

24. The composition according to claim 1, wherein the active component of the composition at least one wetting agent.

25. The composition according to claim 1, wherein, in addition to the active component, the composition further comprises at least one surfactant.

26. The composition according to claim 25, wherein said at least one surfactant is amphoteric.

27. The composition according to claim 25, wherein said at least one surfactant is a mixture of amphoteric surfactants.

28. The composition according to claim 1, wherein the composition comprises between 0.01% and 20% by volume of the active component and between 15% and 80% by volume of at least one surfactant, and water.

29. The composition according to claim 1, wherein the composition comprises between 0.1% and 10% by volume of the active component and between 20% and 60% by volume of at least one surfactant, and water.

30. The composition according to claim 1, wherein the composition comprises substantially 1% by volume of the active component and substantially 44% by volume of at least one surfactant, and water.

31. The composition according to claim 1, wherein the composition is provided in a form ready for immediate delivery to skin.

32. The composition according to claim 1, wherein the composition is provided in an impregnated cloth wipe.

33. The composition according to claim 1, wherein the composition is provided in a solid bar or tablet.

34. A means of destroying organisms and/or inhibiting the ability of bacteria and/or viruses to replicate when said bacteria and/or viruses are present on the skin, the means comprising the application of a composition to said skin wherein the composition is configured to rupture the phospholipid membrane of the bacteria or virus, the composition being further configured to substantially permanently bind the bacterial DNA and viral DNA or RNA.

35. The means according to claim 34, wherein the composition comprises an active component, and wherein the active component comprises at least one alcohol, at least one long-chain alkyl polyamine, and at least one halogen.

36. A means of inhibiting the ability of bacteria and/or viruses to replicate when said bacteria and/or viruses are present on the skin, the means comprising the application of a composition to said skin wherein the composition is configured to substantially permanently encapsulate the bacterial or viral structures and prevent the replication of their genetic material.

37. The means according to claim 36, wherein the composition comprises an active component, and wherein the active component comprises at least one alcohol, at least one long-chain alkyl polyamine, and at least one halogen.

38. A method for manufacturing a skin cleaning and disinfecting composition wherein the method comprises:
the addition to a pH buffered solution of at least one long-chain alkyl polyamine, to which is added at least one surfactant to make an interim solution;
separate to said interim solution a premix solution containing at least one alcohol and at least one halogen is made;
the interim solution and the premix solution are then combined, to produce an active component; and
wherein the active component is subsequently mixed with at least one surfactant.

39. A skin clearing and disinfecting composition comprising an active component, wherein the active component comprises at least one alcohol, at least one long-chain alkyl polyamine.
